(19) United States
(12) Patent Application Publication
(10) Pub. No.: US 2013/0028992 A1
(43) Pub. Date: Jan. 31, 2013

(54) Herbal-Based Solution for Treating Symptoms of Nasal Conditions

(71) Applicant: Caleb James Walker, Columbus, GA (US)

(72) Inventor: Caleb James Walker, Columbus, GA (US)

(21) Appl. No.: 13/190,480

(22) Filed: Jul. 26, 2011

(51) Int. Cl. A61K 36/00; A61P 31/00; A61P 29/00; A61K 31/26; A61P 37/08

(52) U.S. Cl. 424/725; 514/608

(57) ABSTRACT

An herbal-based solution includes a first amount of wasabi comprising at least an allyl isothiocyanate component, and a second amount of saline solution. The saline solution and the organic material are combined to substantially dilute the wasabi in the saline solution to produce the herbal-based solution. The herbal-based solution further has anti-inflammatory, anti-microbial and anti-coagulative properties. The wasabi may include Eutrema Wasabi. The herbal-based solution is deliverable as a nasal.

### FEDERALLY SPONSORED RESEARCH OR DEVELOPMENT

Not applicable.

### REFERENCE TO SEQUENCE LISTING, A TABLE, OR A COMPUTER LISTING APPENDIX

Not applicable.

### COPYRIGHT NOTICE

A portion of the disclosure of this patent document contains material that is subject to copyright protection. The copyright owner has no objection to the facsimile reproduction by anyone of the patent document or patent disclosure as it appears in the Patent and Trademark Office, patent file or records, but otherwise reserves all copyright rights whatsoever.

### FIELD OF THE INVENTION

The present invention relates generally to homeopathic remedies. More particularly, the invention relates to a homeopathic nasal relief solution.

### BACKGROUND OF THE INVENTION

The present invention relates to treatments for the symptoms of sinus and nasal conditions such as, but not limited to, sinusitis, rhinitis, allergies, nasal polyps, etc. These conditions cause many symptoms including, without limitation, sinus congestion and pressure, nasal drip, drying of the nasal passages, headache, etc.

Many treatments for the symptoms of sinus and nasal conditions currently exist. These treatments may be obtained over the counter or with a prescription and include, without limitation, antibiotics, antihistamines, decongestants, leukotrinenes, mucolytics, steroids, etc. Treatments may come in various different forms such as, but not limited to, pills, liquids or nasal sprays. Traditional over-the-counter and prescription sinus medications offer only short-term benefits and may cause side effects including, but not limited to, drowsiness, upset stomach, nausea, headache, dizziness, irregular heart beat, fungal overgrowth in the nasal passages, etc. Some sinus treatments involve the use of steroids; however, these treatments may irritate the nasal passages or cause bleeding. Furthermore, steroidal or medicated treatments may harmfully interact with other medications and may be addictive, and treatments using antibiotics may promote antibiotic resistance. It is therefore an objective of the present invention to provide a treatment for sinus and nasal conditions that does not use antibiotics, steroids or other medications.

The prior art includes treatments that are composed of natural ingredients in an attempt to avoid the side effects of medicated treatments. For example, people have used saline solution in the past to clear and moisturize the lining of the nasal cavity and sinuses. In some cases the saline is combined with other ingredients such as, but not limited to, lavender. Saline solution is typically introduced to the effected area with a spray-mist bottle that sprays the solution up into the nasal passages. One such saline solution used as a nasal spray is made with the capsaicin of peppers. This formula is intended to relieve nasal congestion, runny nose, sneezing, sinus pressure, and headache.

Other known nasal sprays include a spay that uses osmotic effect to help alleviate decongestion, a nasal spray containing a mucolytic agent to shrink mucosa and an herbal based nasal spray to be used as a decongestant and antihistamine. However, these nasal sprays may be addictive or habit forming. Furthermore, frequent use of these nasal sprays may result in rebound congestion, which is when a decongestant becomes ineffective or causes the congestion to worsen.

In view of the foregoing, there is a need for improved techniques for providing a homeopathic treatment for sinus or nasal conditions that is non-habit forming and does not cause rebound congestion.

### BRIEF DESCRIPTION OF THE DRAWINGS

The present invention is illustrated by way of example, and not by way of limitation, in the figures of the accompanying drawings and in which like reference numerals refer to similar elements and in which:

FIG. 1 is a flowchart illustrating an exemplary method for making and using a wasabi saline solution for the treatment of nasal conditions, in accordance with an embodiment of the present invention; and

FIG. 2 is a side view of an exemplary nasal spray-mist bottle being used to administer a wasabi saline solution into nasal passages of a user, in accordance with an embodiment of the present invention.

Unless otherwise indicated illustrations in the figures are not necessarily drawn to scale.

### SUMMARY OF THE INVENTION

To achieve the forgoing and other objects and in accordance with the purpose of the invention, an herbal-based solution for treating symptoms of nasal conditions is presented.

In one embodiment a nasal includes a first amount of organic material comprising at least an allyl isothiocyanate component and a second amount of saline solution. The saline solution and the organic material being combined where the organic material is substantially diluted in the saline solution to produce an herbal-based solution for treating symptoms of nasal conditions. In other embodiments the herbal-based solution comprises anti-inflammatory properties, anti-microbial properties and anti-coagulative properties. In yet another embodiment the organic material comprises wasabi. In still another embodiment the wasabi is in a paste form. In other embodiments the wasabi comprises a liquid or a powder form. In yet another embodiment the wasabi comprises Eutrema Wasabi. In still another embodiment the herbal-based solution is delivered by a nasal spray-mist bottle. In another embodiment the herbal-based solution is deliverable using a neti-pot or neti-bottle. In yet another embodiment the herbal-based solution generally comprises one part organic material to twenty parts saline solution.

In another embodiment a method includes steps of measuring a first amount of an organic material comprising at least an allyl isothiocyanate component and measuring a second amount of a saline solution. The first amount and the second amount are combined. The combined amounts are agitated until the organic material is substantially diluted in the saline solution to produce an herbal-based solution for treating symptoms of nasal conditions. The herbal-based solution delivered in a container for dispensing as a nasal. In another embodiment the organic material comprises wasabi. In yet another embodiment the wasabi comprises Eutrema Wasabi. In still another embodiment the container is a nasal spray-mist bottle for dispensing the nasal as a mist. In another embodiment the container is a neti-pot or neti-bottle for dispensing the nasal as irrigation. In yet another embodiment a ratio of the second amount to the first amount is generally twenty to one.

In another embodiment an herbal-based solution includes a first amount of wasabi comprising at least an allyl isothiocyanate component and a second amount of saline solution. The saline solution and the organic material being combined to substantially dilute the wasabi in the saline solution to produce the herbal-based solution further comprising anti-inflammatory, anti-microbial and anti-coagulative properties. In another embodiment the wasabi comprises Eutrema Wasabi. In yet another embodiment the herbal-based solution is deliverable as a nasal.

Other features, advantages, and objects of the present invention will become more apparent and be more readily understood from the following detailed description, which should be read in conjunction with the accompanying drawings.

### DETAILED DESCRIPTION OF THE PREFERRED EMBODIMENTS

The present invention is best understood by reference to the detailed figures and description set forth herein.

Embodiments of the invention are discussed below with reference to the Figures. However, those skilled in the art will readily appreciate that the detailed description given herein with respect to these figures is for explanatory purposes as the invention extends beyond these limited embodiments. For example, it should be appreciated that those skilled in the art will, in light of the teachings of the present invention, recognize a multiplicity of alternate and suitable approaches, depending upon the needs of the particular application, to implement the functionality of any given detail described herein, beyond the particular implementation choices in the following embodiments described and shown. That is, there are numerous modifications and variations of the invention that are too numerous to be listed but that all fit within the scope of the invention. Also, singular words should be read as plural and vice versa and masculine as feminine and vice versa, where appropriate, and alternative embodiments do not necessarily imply that the two are mutually exclusive.

It is to be further understood that the present invention is not limited to the particular methodology, compounds, materials, manufacturing techniques, uses, and applications, described herein, as these may vary. It is also to be understood that the terminology used herein is used for the purpose of describing particular embodiments only, and is not intended to limit the scope of the present invention. It must be noted that as used herein and in the appended claims, the singular forms “a,” “an,” and “the” include the plural reference unless the context clearly dictates otherwise. Thus, for example, a reference to “an element” is a reference to one or more elements and includes equivalents thereof known to those skilled in the art. Similarly, for another example, a reference to “a step” or “a means” is a reference to one or more steps or means and may include sub-steps and subservient means. All conjunctions used are to be understood in the most inclusive sense possible. Thus, the word “or” should be understood as having the definition of a logical “or” rather than that of a logical “exclusive or” unless the context clearly necessitates otherwise. Structures described herein are to be understood also to refer to functional equivalents of such structures. Language that may be construed to express approximation should be so understood unless the context clearly dictates otherwise.

Unless defined otherwise, all technical and scientific terms used herein have the same meanings as commonly understood by one of ordinary skill in the art to which this invention belongs. Preferred methods, techniques, devices, and materials are described, although any methods, techniques, devices, or materials similar or equivalent to those described herein may be used in the practice or testing of the present invention. Structures described herein are to be understood also to refer to functional equivalents of such structures. The present invention will now be described in detail with reference to embodiments thereof as illustrated in the accompanying drawings.

From reading the present disclosure, other variations and modifications will be apparent to persons skilled in the art. Such variations and modifications may involve equivalent and other features which are already known in the art, and which may be used instead of or in addition to features already described herein.

Although Claims have been formulated in this application to particular combinations of features, it should be understood that the scope of the disclosure of the present invention also includes any novel feature or any novel combination of features disclosed herein either explicitly or implicitly or any generalization thereof, whether or not it relates to the same invention as presently claimed in any Claim and whether or not it mitigates any or all of the same technical problems as does the present invention.

Features which are described in the context of separate embodiments may also be provided in combination in a single embodiment. Conversely, various features which are, for brevity, described in the context of a single embodiment, may also be provided separately or in any suitable subcombination. The Applicants hereby give notice that new Claims may be formulated to such features and/or combinations of such features during the prosecution of the present application or of any further application derived therefrom.

As is well known to those skilled in the art many careful considerations and compromises typically must be made when designing for the optimal manufacture of a commercial implementation any system, and in particular, the embodiments of the present invention. A commercial implementation in accordance with the spirit and teachings of the present invention may configured according to the needs of the particular application, whereby any aspect(s), feature(s), function(s), result(s), component(s), approach(es), or step(s) of the teachings related to any described embodiment of the present invention may be suitably omitted, included, adapted, mixed and matched, or improved and/or optimized by those skilled in the art, using their average skills and known techniques, to achieve the desired implementation that addresses the needs of the particular application.

It is to be understood that any exact measurements/dimensions or particular construction materials indicated herein are solely provided as examples of suitable configurations and are not intended to be limiting in any way. Depending on the needs of the particular application, those skilled in the art will readily recognize, in light of the following teachings, a multiplicity of suitable alternative implementation details.

A preferred embodiment of the present invention provides an herbal-based solution to be used as a supplement to traditional methods of relieving the symptoms of nasal conditions such as, but not limited to, nasal rhinitis and sinusitis. In some preferred embodiments the solution is made to be used in a nasal spray-mist bottle, or a neti-bottle or pot. An ingredient included in many preferred embodiments is wasabi. The nasal solution can be made out of pure wasabi japonica ingredients or from a mixture of seiyo wasabi (horseradish, mustard seeds and parts of real wasabi). In some embodiments a commercially available wasabi, in the form of a paste, was included. The nutrition information for this paste is shown in Table 1.

TABLE 1

Amount Per Serving

| | |
|---|---|
| Calories | 15 |
| Calories From Fat | 5 |

| | | | % Daily Value |
|---|---|---|---|
| Total Fat | 0.5 | g | 1% |
| Saturated Fat | 0 | g | 0% |
| Trans Fat | 0 | g | 0% |
| Cholesterol | 0 | mg | 0% |
| Sodium | 100 | mg | 4% |
| Total | 3 | g | 1% |
| Carbohydrate | | | |
| Dietary Fiber | 0 | g | 0% |
| Sugar | 0 | g | |
| Protein | 0 | g | |
| Vitamin A | | | 0% |
| Vitamin C | | | 0% |
| Calcium | | | 0% |
| Iron | | | 0% |

The chemicals in fresh Eutrema Wasabi (Japanese wasabi plant) are typically applied in food uses; however, these chemicals may also have medical benefits. The characteristics of Hon-wasabi root (rhizome, raw), (Real Wasabi ingredients) (edible part: 100 g, without leaf stem and lateral root base), are shown in Table 2.

TABLE 2

| | | | | | |
|---|---|---|---|---|---|
| Calorie | 88 | kcal | | | |
| Water | 74.2 | g | | | |
| Protein | 5.6 | g | | | |
| Total lipid | 0.2 | g | Vitamin A (β-carotene) | 7 | μg |
| (fat) | | | Vitamin E (α-tocopherol) | 1.4 | mg |
| Carbohydrate | 18.4 | g | Vitamin K | 49 | μg |
| Ash | 1.5 | g | Vitamin B1 (thiamin) | 0.06 | mg |
| Dietary fiber | 4.4 | g | Vitamin B2 (riboflavin) | 0.15 | mg |
| (soluble) | (0.8 | g) | Niacin (nicotinic acid) | 0.6 | mg |
| (insoluble) | (3.6 | g) | Vitamin B6 | 0.32 | mg |
| Cholesterol | 0 | mg | Folic acid | 50 | μg |
| Sodium (Na) | 24 | mg | Pantothenic acid | 0.2 | mg |
| Potassium (K) | 500 | mg | Vitamin C (ascorbic acid) | 75 | mg |
| Calcium (Ca) | 100 | mg | | | |
| Magnesium | 46 | mg | | | |
| (Mg) | | | | | |
| Phosphorus (P) | 79 | mg | | | |
| Iron (Fe) | 0.8 | mg | | | |
| Zinc (Zn) | 0.7 | mg | | | |
| Copper (Cu) | 0.03 | mg | | | |
| Manganese (Mn) | 0.14 | mg | | | |
| Salt equiv. | 0.1 | g | | | |

Biocidally active chemicals in wasabi are said to act as an antidote to food poisoning, which is one factor that may have led to the use of wasabi with raw fish dishes in Japan. Hydration of a glucoside (sinigrin) by the enzyme myrosinase results in the production of an allyl isothiocyanate, which is the major pungent component in wasabi that causes heat to rush up a person's nose when eating wasabi. The components that give wasabi its flavor are 6-methylthiohexyl isothiocyanate, 7-methylthioheptyl isothiocyanate and 8-methylthioocytl isothiocyanate. Scientists are now discovering that these wasabi isothiocyanates may have important medical benefits.

Recent research shows that certain isothiocyanates from wasabi have potent antibacterial action against the microbes Staphylococcus aureus and Escherichia coli. Research also shows that the vapor from allyl isothiocyanate from wasabi can combat 25 strains of yeast, bacteria and mold to varying degrees. Researchers also contend that the isothiocyanates in wasabi not only inhibit microbes, but can also help treat or prevent blood clotting, asthma and cancer. Wasabi has even been known to prevent tooth decay by inhibiting the growth of Streptococcus mutans, the bacteria that causes dental cavities, during test-tube studies. This is due to wasabi's ability to interfere with the sucrose-dependent adherence of cells. Testing done regarding isothiocyanates found in wasabi on anticoagulative effects including the inhibition of platelet aggregation and platelet deaggregation has found that in the case of a heart attack, where aspirin is commonly prescribed, the isothiocyanates in wasabi have an immediate effect as opposed to the thirty minutes typically required for aspirin to take effect. This anti-inflammatory effect may potentially be used to counter conditions such as, but not limited to, asthma or even anaphylaxis and may reduce inflammation in the nasal passages due to sinus and nasal conditions. Research undertaken regarding isothiocyanates in wasabi and anti-cancer properties has found that some isothiocyanates play a protective role against breast, stomach and colon cancers and that human stomach cancer cells undergo morphological alterations and many die when cultured in media containing a concentration of an aqueous extract from wasabi.

Embodiments of the present invention help to clear-up nasal conditions and also help by killing microbes and pathogens generally preventing likelihood of future health problems.

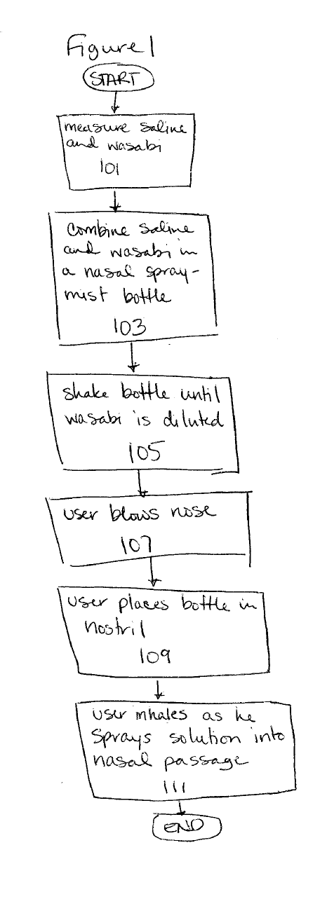

FIG. 1 is a flowchart illustrating an exemplary method for making and using a wasabi saline solution for the treatment of nasal conditions, in accordance with an embodiment of the present invention. In the present embodiment, the solution comprises standard saline and a small percentage of wasabi. The preferred method of making the wasabi saline solution begins by measuring the saline and wasabi in step 101. To achieve the preferred percentage of wasabi to saline, 0.5 mL of wasabi is measured for every 10 mL of saline. Those skilled in the art will readily recognize, in accordance with the teachings of the present invention, that the percentage of wasabi to saline used in alternate embodiments may vary. In the present embodiment, wasabi liquid is preferably used as it easily combines with the saline; however, different types of wasabi may be used in alternate embodiments such as, but not limited to, wasabi powder or wasabi paste. Depending on the type of ingredients being used, different types of measuring devices may be used. For example, a measuring syringe is preferably used to measure the saline and liquid wasabi; however, measuring beakers, graduated cylinders or other volume measuring devices may also be used. Furthermore, if wasabi paste or powder is being used, other types of measuring devices may be used such as, but not limited to, scales, measuring cups or measuring spoons. Once the ingredients are measured, they are combined in a nasal spray-mist bottle in step 103. In step 105, the saline and wasabi in the bottle are agitated by shaking the bottle or stirring the solution until the wasabi is completely diluted into the saline. The solution is now ready for use.

In typical use of the present invention, a user first clears his airway by blowing his nose into a tissue in step 107. The user then places the nozzle of the bottle in a nostril in step 109, and in step 111 inhales while squeezing the misting trigger on the bottle to spray the solution up into his nasal passage. Those skilled in the art will readily recognize, in accordance with the teachings of the present invention, that any of the foregoing steps may be suitably replaced, reordered, removed and additional steps may be inserted depending upon the needs of the particular application. For example, without limitation, the ingredients may be measured and placed into the bottle separately from each other, or the user may skip the step of blowing his nose before spraying the solution into his nasal passage.

In the present embodiment, the wasabi saline solution provides relief from severe nasal congestion and nasal drip that may be caused by nasal conditions such as, but not limited to, sinusitis, rhinitis or allergies. The solution also provides moisture to the nasal passages and generally prevents bacteria and mold from growing in the nasal passages and causing problems in the nasal passages and sinuses. The solution cleans the nasal passages of crusts, mucus and other blockages and also helps in the natural cleaning of the nasal passages. The anti-inflammatory properties of the wasabi may also help to relieve sinus pressure and to shrink nasal polyps. Various different types of people may use the wasabi saline solution according to the present invention including, without limitation, people with recurring nasal-sinus discomfort, people with dry, sensitive nasal tissue, people who are exposed to pollen, dust, mold or high humidity, people who travel by airplane or other mass transit, people who have school-age children, people who work in or frequently visit a hospital, people who are frequently among crowds, people who are concerned about long-term use of medicated sprays, people with allergies, etc. Furthermore, with the known cancer fighting properties of one of the compounds found in wasabi, the wasabi saline solution may be able to help fight cancer cells.

In the present embodiment, the wasabi saline solution is herbal and does not contain any chemicals. The solution does not have any side effects, is non-habit forming, non-drowsy, and moisture-rich. The solution provides hygienic nasal-sinus relief. The use of the present embodiment and simple, daily nasal cleansing can provide a lifetime of nasal-sinus relief and comfort. Unlike many decongestant nasal sprays, using the wasabi saline solution according to the present embodiment does not lead to nasal spray addiction or rebound congestion. The solution is non-steroidal, does not promote antibiotic resistance, and has no known drug interactions. The solution is safe for many users including, without limitation, adults, children 6 years and older, diabetics, pregnant or lactating women, hypertensive individuals, etc.

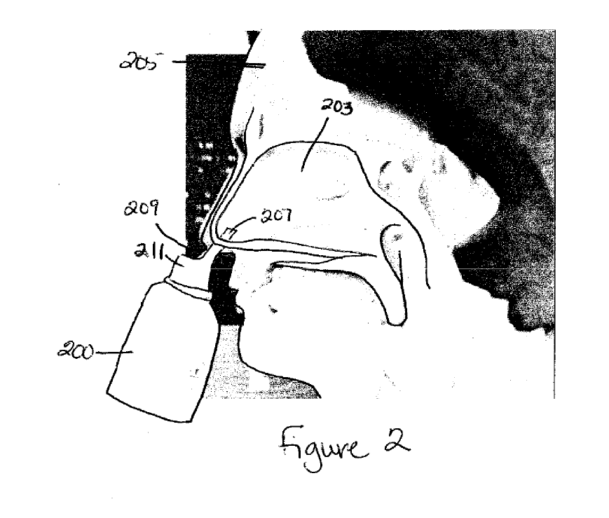

FIG. 2 is a side view of an exemplary nasal spray-mist bottle 200 being used to administer a wasabi saline solution into nasal passages 203 of a user 205, in accordance with an embodiment of the present invention. In typical use of the present embodiment, user 205 inserts a nozzle 207 of bottle 200 into a nostril 209. User 205 then inhales as he squeezes a misting trigger 211 on bottle 200 to spray the solution in bottle 200 into nasal passages 203. In alternate embodiments, different types of bottles may be used to introduce the solution into the nasal passages such as, but not limited to, non-misting bottles, bottles with pumps rather than triggers, aerosol bottles, etc.

Having fully described at least one embodiment of the present invention, other equivalent or alternative methods of providing a homeopathic solution for treating nasal and sinus conditions according to the present invention will be apparent to those skilled in the art. The invention has been described above by way of illustration, and the specific embodiments disclosed are not intended to limit the invention to the particular forms disclosed. For example, the particular implementation of the solution may vary depending upon the particular type of delivery method used. The delivery methods described in the foregoing were directed to spray bottle implementations; however, similar techniques are to deliver the solution to the nasal passages using various different means such as, but not limited to, steam, nasal irrigation systems, syringes, swabs, etc. Non-spray bottle implementations of the present invention are contemplated as within the scope of the present invention. The invention is thus to cover all modifications, equivalents, and alternatives falling within the spirit and scope of the following claims.

Claim elements and steps herein may have been numbered and/or lettered solely as an aid in readability and understanding. Any such numbering and lettering in itself is not intended to and should not be taken to indicate the ordering of elements and/or steps in the claims.

What is claimed is:

1. A nasal comprising:
a first amount of organic material comprising at least an allyl isothiocyanate component; and
a second amount of saline solution, said saline solution and said organic material being combined where said organic material is substantially diluted in said saline solution to produce a herbal-based solution for treating symptoms of nasal conditions.

2. The nasal as recited in claim 1, in which said herbal-based solution comprises anti-inflammatory properties.

3. The nasal as recited in claim 1, in which said herbal-based solution comprises anti-microbial properties.

4. The nasal as recited in claim, in which said herbal-based solution comprises anti-coagulative properties.

5. The nasal as recited in claim 1, in which said organic material comprises wasabi.

6. The nasal as recited in claim 5, in which said wasabi is in a paste form.

7. The nasal as recited in claim 5, in which said wasabi comprises a liquid or a powder form.

8. The nasal as recited in claim 5, in which said wasabi comprises Eutrema Wasabi.

9. The nasal as recited in claim 1, in which said herbal-based solution is delivered by a nasal spray-mist bottle.

10. The nasal as recited in claim 1, in which said herbal-based solution is deliverable using a neti-pot or neti-bottle.

11. The nasal as recited in claim 1, in which said herbal-based solution generally comprises one part organic material to twenty parts saline solution.

12. A method comprising steps of:
measuring a first amount of an organic material comprising at least an allyl isothiocyanate component;
measuring a second amount of a saline solution;
combining said first amount and said second amount;
agitating said combined amounts until said organic material is substantially diluted in said saline solution to produce a herbal-based solution for treating symptoms of nasal conditions; and
delivering said herbal-based solution in a container for dispensing as a nasal.

13. The method as recited in claim 12, in which said organic material comprises wasabi.

14. The method as recited in claim 13, in which said wasabi comprises Eutrema Wasabi.

15. The method as recited in claim 12, in which said container is a nasal spray-mist bottle for dispensing said nasal as a mist.

16. The method as recited in claim 12, in which said container is a neti-pot or neti-bottle for dispensing said nasal as irrigation.

17. The method as recited in claim 12, in which a ratio of said second amount to said first amount is generally twenty to one.

18. A herbal-based solution comprising:
a first amount of wasabi comprising at least an allyl isothiocyanate component; and
a second amount of saline solution, said saline solution and said organic material being combined to substantially dilute said wasabi in said saline solution to produce the herbal-based solution further comprising anti-inflammatory, anti-microbial and anti-coagulative properties.

19. The herbal-based solution as recited in claim 18, in which said wasabi comprises Eutrema Wasabi.

20. The herbal-based solution as recited in claim 18, in which the herbal-based solution is deliverable as a nasal.
